(19) United States
(12) Patent Application Publication
(10) Pub. No.: US 2019/0221300 A1
(43) Pub. Date: Jul. 18, 2019

(54) METHOD FOR DISPENSING MEDICATION

(71) Applicant: George James Pate, Saint Petersburg, FL (US)

(72) Inventor: George James Pate, Saint Petersburg, FL (US)

(21) Appl. No.: 16/252,568

(22) Filed: Jan. 18, 2019

Related U.S. Application Data
(60) Provisional application No. 62/619,022, filed on Jan. 18, 2018.

(51) Int. Cl. G16H 20/13; A61J 7/00; A61M 5/178

(52) U.S. Cl. CPC G16H 20/13; A61M 5/24; A61M 5/1782; A61J 7/0076

(57) ABSTRACT

Implementations of a method for dispensing one or more tubes of medication from a medication dispensing device are provided. In some implementations, the medication dispensing device comprises a processing system and a dispensing mechanism. In some implementations, the medication dispensing device comprises a data input, a medication input, a medication output, and a billing information output. In some implementations, the method for dispensing one or more tubes of medication from a medication dispensing device comprises receiving medication treatment information, loading a tube of medication in the medication dispensing device, opening the tube of medication, dispensing a needed amount of the medication into a sterile syringe, dispensing the sterile syringe of medication from the medication dispensing device, and providing billing information for the dispensed medication.

### CROSS REFERENCE TO RELATED APPLICATION

This application claims the benefit of U.S. Patent Application Ser. No. 62/619,022, which was filed on Jan. 18, 2018, and is incorporated herein by reference in its entirety.

### TECHNICAL FIELD

This disclosure relates to implementations of a method for dispensing a tube of medication from a medication dispensing device.

### BACKGROUND

In hospitals and other treatment centers, medication is often dispensed from tubes, also known as squeeze tubes, or similar containers for treatment of patients. For example, medications for wound care treatment, such as ointments or creams, are often dispensed from tubes for patients. The tubes of such medication are originally sealed and unopened before dispensing to ensure the medication is sanitary.

However, after a tube is initially opened and the medication from the tube is dispensed to treat a patient, the tube is compromised and must be discarded and thrown away. Although, there are usually additional doses of the medication remaining in the tube after a tube is initially opened and the medication from the tube is dispensed to treat a patient, to ensure the medication dispensed from the tube is sanitary, any such remaining medication in the tube can not be used subsequently for another patient. This waste of such remaining medication increases the cost of patient treatment, including for treatment centers and for Medicare or other payers, and reduces the supply of available medication.

### BRIEF DESCRIPTION OF THE DRAWINGS

FIG. 1 illustrates an implementation of an example environment for performing a method for dispensing a tube of medication from a medication dispensing device according to the present disclosure.

FIG. 2 illustrates a flow diagram of an example implementation of the method for dispensing a tube of medication from a medication dispensing device according to the present disclosure.

FIG. 3 illustrates an example computer system that may be used with some implementations of the present disclosure.

### DETAILED DESCRIPTION

Implementations of a method for dispensing one or more tubes of medication from a medication dispensing device are provided.

In some implementations, the medication dispensing device comprises a processing system and a dispensing mechanism. In some implementations, the medication dispensing device comprises a data input, a medication input, a medication output, and a billing information output.

In some implementations, the medication dispensing device (“dispenser”) is configured to dispense a squeeze tube or “tube” type container of medication (“tube of medication”) for treatment of a wound or other injury while maintaining the sanitary, sterile, and/or otherwise uncompromised condition of the medication.

In some implementations, the dispenser is configured to receive medication treatment information, such as a dosage amount, treatment area (e.g., wound area) information, and/or any other suitable information with respect to the tube of medication and/or the medication treatment.

In some implementations, the dispenser is configured to dispense the tube of medication based on the received medication treatment information.

In some implementations, the dispenser is configured to receive the medication treatment or other information from a Pyxis™ or other medication coordination device.

In some implementations, the dispenser is configured to provide billing information for the medication dispensed from the dispenser. In some implementations, the dispenser is configured to provide the billing information to Medicare, another insurance provider, or any other suitable payer or recipient.

In some implementations, the method for dispensing a tube of medication from a medication dispensing device (“dispenser”) comprises receiving medication treatment information, such as the information described above.

In some implementations, the method comprises loading a tube of medication in the dispenser.

In some implementations, the method comprises opening the tube of medication in the dispenser.

In some implementations, the method comprises dispensing a needed amount of medication from the tube into a sterile syringe. In some implementations, the needed amount of medication dispensed from the tube is based on the received medication treatment information.

In some implementations, the method comprises dispensing the sterile syringe of medication from the dispenser for treatment use.

In some implementations, the method comprises providing billing information for the dispensed medication.

In some implementations, the billing information is provided to Medicare or other insurance (e.g., medical insurance) provider. In some implementations, the billing information is provided to any other suitable payer or recipient.

In some implementations, the method for dispensing medication provides the foregoing features and steps while keeping the medication sanitary, sterile, and/ or otherwise uncompromised.

In some implementations, the method for dispensing medication provides the foregoing features and steps while dispensing from a tube of remaining medication that was previously opened and dispensed and also while keeping the medication sanitary, sterile, and/or otherwise uncompromised.

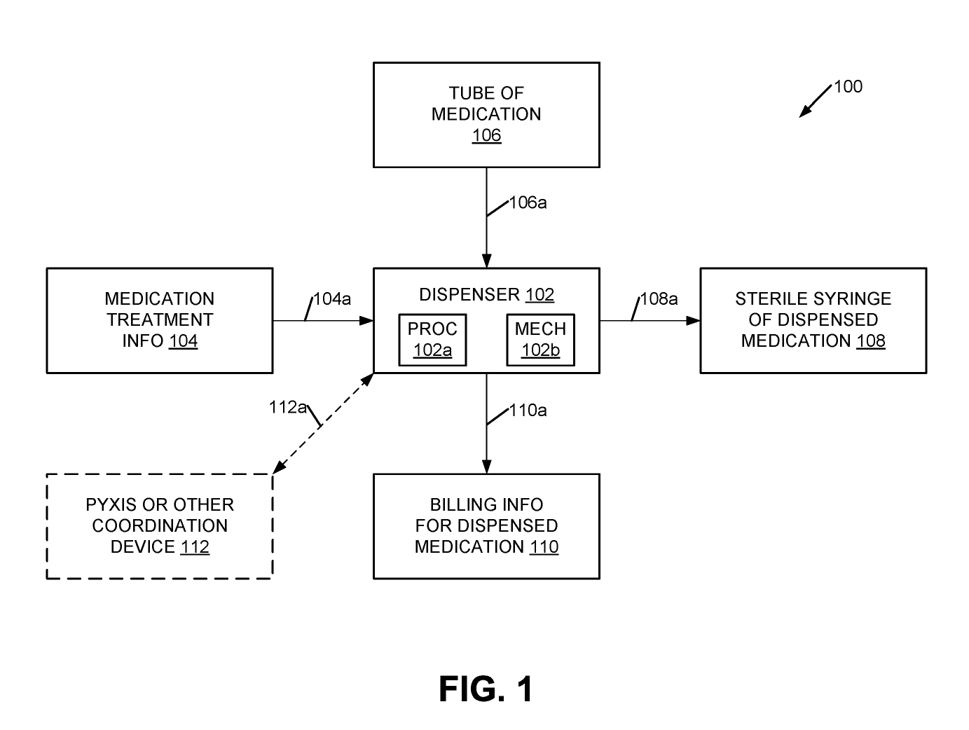

FIG. 1 illustrates an implementation of an example environment 100 for performing a method for dispensing a tube of medication 106 from a medication dispensing device (“dispenser”) 102 according to the present disclosure.

In some implementations, the dispenser 102 comprises a processing system 102a and a dispensing mechanism 102b. In some implementations, the dispenser 102 comprises a data input 104a, a medication input 106a, a medication output 108a, and a billing information output 110a.

In some implementations, the dispenser 102 may comprise additional elements and features, some of which are discussed below, such as a communication connection 112a to a Pyxis™ or other medication coordination device 112.

In some implementations, the processing system 102a is configured to process operations of the dispenser 102, such as described below. In some implementations, the processing system 102a may be any suitable processing system, computer system, or other component(s) capable of processing the operations of the dispenser 102 as described below.

In some implementations, the dispensing mechanism 102b is configured to perform operations of the dispenser 102, such as described below. In some implementations, the dispensing mechanism 102b may be any suitable mechanism, system, or other component(s) capable of performing the operations of the dispenser 102 as described below.

In some implementations, the data input 104a is configured to input medication treatment information 104 to the dispenser 102, such as described below. In some implementations, the data input 104a may be any suitable component(s) capable of inputting the medication treatment information 104 to the dispenser 102 as described below. In some implementations, the data input 104a may be part of the processing system 102a.

In some implementations, the medication treatment information 104 comprises information about a wound to be treated by the tube of medication, including the type of the wound, the size of the wound, and/or the condition of the wound.

In some implementations, the data input 104a is connected to a Pyxis™ or other medication coordination device 110 that inputs the medication treatment information 104 to the dispenser 102 through the data input 104a.

In some implementations, the medication input 106a is configured to input one or more tubes of medication 106 to the dispenser 102, such as described below. In some implementations, the medication input 106a may be any suitable mechanism and/ or other component(s) capable of inputting one or more tubes of medication 106 to the dispenser 102 as described below. In some implementations, the medication input 106a may be part of the dispensing mechanism 102b.

In some implementations, the tube of medication 106 comprises a wound or other injury treatment ointment or cream.

In some implementations, the medication output 108a is configured to output a determined amount of the medication from the dispenser 102 in a sterile syringe 108, such as described below. In some implementations, the medication output 108a may be any suitable mechanism and/or other component(s) capable of outputting the determined amount of the medication from the dispenser 102 in a sterile syringe 108 as described below. In some implementations, the medication output 108a may be part of the dispensing mechanism 102b.

In some implementations, the billing information output 110a is configured to output billing information 110 based on the amount of medication 104 that is dispensed from the dispenser 102 in the sterile syringe 108, such as described below. In some implementations, the billing information output 110a may be any suitable component(s) capable of outputting the billing information 110 as described below. In some implementations, the billing information output 110a may be part of the processing system 102a.

In some implementations, the billing information output 110a is configured to transmit the billing information 110 to or output the billing information 110 to submit to Medicare or another medical insurance provider.

In some implementations, the dispenser 102 is configured to receive medication treatment information 104 through the data input 104a by a processing of the processing system 102a.

In some implementations, the dispenser 102 is configured to receive medication treatment information 104, such as a dosage amount, treatment area (e.g., wound area) information, and/or any other suitable information with respect to the tube of medication 106 and/or the medication treatment.

In some implementations, medication treatment information 104 may be information regarding the size of a wound or other injury, such as a burn or laceration, of a patient for treatment. In some implementations, medication treatment information 104 may be a general description of the treatment area such as small, medium, or large.

In some implementations, medication treatment information 104 may be the dimensions of the treatment area, such as the length and width of the treatment area. In some implementations, such dimensions of the treatment area may include the depth of the treatment area.

In some implementations, such dimensions of the treatment area may include any other suitable measurement of the treatment area to provide medication treatment information 104 to the dispenser 102 to determine the amount of medication 106 to dispense. In some implementations, such dimensions of the treatment area may be approximate.

In some implementations, the dispenser 102 may be configured to receive any other suitable information 104 for dispensing medication 106 for treatment of a patient, such as treatment of a wound or any other medicinal treatment. In some implementations, such other suitable information 104 may include wound or treatment area type, wound or treatment area condition, wound or treatment area severity, relevant patient information such as medication allergies, and/or the type of medication needed for treatment of the treatment area.

In some implementations, the dispenser 102 may be configured to operate with a Pyxis™ or other medication coordination device 112.

In some implementations, the dispenser 102 may be configured to receive the medication treatment information 104 by a processing of the processing system 102a through the data input 104a from a Pyxis™ or other medication coordination device 112 connected to the data input 104a.

As shown in FIG. 1, in some implementations, the dispenser 102 may be configured to connect and interface with the Pyxis™ or other medication coordination device 112. For example, in some implementations, the Pyxis™ or other medication coordination device 112 may be connected to the dispenser 102 by a communication connection 112a. Alternately, although not depicted, in some implementations, the Pyxis™ or other medication coordination device 112 may be connected to the dispenser 102 through the data input 104a as described above.

In some implementations, the dispenser 102 may be configured to transmit or receive data from the Pyxis™ or other medication coordination device 112. In some implementations, the dispenser 102 may be configured to be coordinated or controlled by the Pyxis™ or other medication coordination device 112.

In some implementations, the communication connection 112a may be a wired or a wireless connection. In some implementations, the communication connection 112a may be any other suitable connection.

In some implementations, the communication connection 112a may be configured to transmit data and/or control inputs and/or outputs between the dispenser 102 and the Pyxis™ or other medication coordination device 112. In some implementations, the communication connection 112a may be configured to transmit any other suitable communications between the dispenser 102 and the Pyxis™ or other medication coordination device 112.

In some implementations, the dispenser 102 is configured to load or be loaded with a tube of medication 106.

In some implementations, the dispenser 102 is configured to load the tube of medication 106 through the medication input 106a by an operation of the dispensing mechanism 102b.

In some implementations, the dispenser 102 is configured to house or otherwise hold and/or contain one or more tubes of medication 106.

In some implementations, the dispenser 102 may be configured to load a plurality of tubes of medication 106 that contain the same or different medications 106.

In some implementations, the tube of medication 106 may contain medication for treatment of a wound or other injury of a patient. For example, in some implementations, the tube of medication 106 may contain a treatment ointment or cream for treatment of a wound or other injury of a patient.

In some implementations, the tube of medication 106 may contain any other suitable medication for treatment of a wound or other injury of a patient. In some implementations, the tube of medication 106 may contain medication for any other medicinal treatment.

In some implementations, the tube of medication 106 may originally be sealed and unopened.

In some implementations, the medication in the tube 106 may be sanitary, sterile, and/or otherwise uncompromised.

In some implementations, the dispenser 102 is configured to open the tube of medication 106. In some implementations, the dispenser 102 is configured to open the tube of medication 106 by an operation of the dispensing mechanism 102b.

In some implementations, the dispenser 102 is configured to open the tube of medication 106 while keeping the medication sanitary, sterile, and/or otherwise uncompromised.

In some implementations, the dispenser 102 is configured to keep the medication sanitary, sterile, and/or otherwise uncompromised after the tube of medication 106 has been opened but not used to dispense the medication. In some implementations, the dispenser 102 is configured to keep the medication sanitary, sterile, and/or otherwise uncompromised after the tube of medication 106 has been opened and used to dispense the medication.

In some implementations, the dispenser 102 is configured to determine by a processing of the processing system 102a the amount of the medication 106 to dispense from the tube of medication 106 into the sterile syringe 108 based on the medication treatment information 104.

In some implementations, the dispenser 102 is configured to dispense a desired amount of medication 106 from the loaded tube of medication 106 into a sterile syringe 108. In some implementations, the dispenser 102 is configured to dispense the needed amount of medication 106 for treatment of a wound, other injury, or any other medicinal treatment of a patient based on input information.

In some implementations, the dispenser 102 is configured to dispense a desired amount of medication 106 during or at a desired time prior to the treatment of a wound, other injury, or any other medicinal treatment of a patient.

In some implementations, the dispenser 102 is configured to dispense the desired amount of medication 106 based on the information 104 received by (e.g., inputted to) the dispenser 102.

In some implementations, the dispenser 102 is configured to dispense a desired amount of medication 106 based on a dosage amount. In some implementations, the dispenser 102 is configured to dispense a desired amount of medication based on the medication treatment information 104.

In some implementations, the dispenser 102 may be configured to dispense a desired amount of medication 106 determined based on any other suitable information for dispensing medication 106.

In some implementations, the dispenser 102 is configured to dispense a desired amount of medication from the loaded tube of medication 106 to the sterile syringe 108 while keeping the medication sterile, sanitary, and/or otherwise uncompromised.

In some implementations, the dispenser 102 is configured to keep any remaining medication 106 that is in the tube sterile, sanitary, and/or otherwise uncompromised after the desired amount of medication 106 is dispensed to the sterile syringe 108.

In some implementations, the dispenser 102 is configured to dispense a desired amount of medication to the sterile syringe 108 from a tube of remaining medication 106 that was previously opened and dispensed by the dispenser 102 while keeping the medication sterile, sanitary, and/or otherwise uncompromised.

In some implementations, the sterile syringe 108 may be any other suitable sterile applicator of medication 106 for treatment of a wound, other injury, or any other medicinal treatment.

In some implementations, the dispenser 102 is configured to dispense a sterile syringe 106 of medication 108.

In some implementations, the dispenser 102 is configured to dispense the determined amount of the medication 106 from the tube of medication 106 into the sterile syringe 108 by an operation of the dispensing mechanism 102b.

In some implementations, the dispenser 102 is configured to dispense the sterile syringe 108 containing the determined amount of the medication 106 from the dispenser 102 through the medication output 108a by an operation of the dispensing mechanism 102b.

In some implementations, the dispenser 102 is configured to dispense the sterile syringe of medication 108 for use in the treatment of a wound or other injury of a patient. In some implementations, the dispenser 102 is configured to dispense the sterile syringe of medication 108 for any other medicinal purpose.

In some implementations, the dispenser 102 is configured to dispense the sterile syringe of medication 108 while keeping the medication sterile, sanitary, and/or otherwise uncompromised.

In some implementations, the dispenser 102 may be configured to dispense the sterile syringe of dispensed medication 108 during or at a desired time prior to the treatment.

In some implementations, the dispenser 102 is configured to determine the billing information 110 by a processing of the processing system 102a based on the amount of the medication 106 from the tube of medication 106 that is dispensed from the dispenser 102 in the sterile syringe 108.

In some implementation, the dispenser 102 is configured to output billing information 110 for the dispensed medication 106.

In some implementations, the dispenser 102 is configured to provide billing information 110 for the dispensed medication 106.

In some implementations, the dispenser 102 is configured to provide the billing information 110 based on the dispensed amount of medication 106 through the billing information output 110a by a processing of the processing system 102a.

In some implementations, the billing information 110 comprises a billing payment amount, such as a cost billed to a patient, for the dispensed amount of medication 106 in the sterile syringe 108.

In some implementations, the dispenser 102 is configured to provide the billing information 110 for submission to Medicare or other insurance (e.g., medical insurance) provider. In some implementations, the dispenser 102 is configured to provide the billing information 110 to Medicare or another medical insurance provider by a processing of the processing system 102a through the billing information output 110a.

In some implementations, the dispenser 102 is configured to provide the billing information 110 for submission to any other suitable payer or recipient.

In some implementations, the dispenser 102 is configured to transmit the billing information 110 to Medicare or other insurance provider. In some implementations, the dispenser 102 is configured to transmit the billing information 110 to any other suitable payer or recipient.

In some implementations, the dispenser 102 is configured to access networks to transmit information (such as billing information) to servers (such as a payer or any other recipient). In some implementations, the dispenser 102 can connect to a network through a wireless cellular network, such as GPRS-based and CDMA-based wireless networks, as well as 802.16 WiMax and long-range wireless data networks.

The network may be any suitable network. In some implementations, for example, one or more portions of the network may include an ad hoc network, an intranet, an extranet, a virtual private network (VPN), a local area network (LAN), a wireless LAN (WLAN), a wide area network (WAN), a wireless WAN (WWAN), a metropolitan area network (MAN), a portion of the Internet, a portion of the Public Switched Telephone Network (PSTN), a cellular telephone network, another network, or a combination of two or more of the foregoing.

In some implementations, the dispenser 102 may be configured to communicate over a link. The link may connect the dispenser 102 to a networks or any other device. In some implementations, one or more links may include one or more wireline (such as for example Digital Subscriber Line (DSL) or Data Over Cable Service Interface Specification (DOCSIS)), wireless (such as for example Wi-Fi or Worldwide Interoperability for Microwave Access (WiMAX)), or optical (such as for example Synchronous Optical Network (SONET) or Synchronous Digital Hierarchy (SDH)) links. In particular embodiments, one or more links 150 may each include an ad hoc network, an intranet, an extranet, a VPN, a LAN, a WLAN, a WAN, a WWAN, a MAN, a portion of the Internet, a portion of the PSTN, a cellular technology-based network, a satellite communications technology-based network, another link, or a combination of two or more such links.

In some implementations, the dispenser 102 is configured to keep the medication 106 sanitary, sterile, and/or otherwise uncompromised while the tube of medication 106 is loaded and opened and the medication 106 is dispensed by an operation of the dispensing mechanism 102b.

In some implementations, the dispenser 102 is configured to keep a remaining portion of the medication 106 that is in the tube of medication 106 sanitary, sterile, and/or otherwise uncompromised by an operation of the dispensing mechanism 102b. In some implementations, the remaining portion of the medication 106 is kept sanitary, sterile, and/or otherwise uncompromised for another dispensing of the medication 106 while the tube of medication 106 remains loaded and opened in the dispenser 102 after the dispensing of the determined amount of the medication 106 from the tube of medication 106 into the sterile syringe 108.

In some implementations, the dispenser 102 may be computer-implemented or computer-controlled. For example, as described throughout above and below, in some implementations, the dispenser 102 comprises a processing system 102a that is configured to process operations of the dispenser 102.

In some implementations, the dispenser 102 may be implemented or controlled by any other suitable manner.

In some implementations, information received by and/or provided from the dispenser 102, such as medication treatment information 104 and/or billing information 110, can be in any suitable form or format.

One of ordinary skill in the art with the benefit of this disclosure would know how to make and use a dispenser 102 according to the present disclosure.

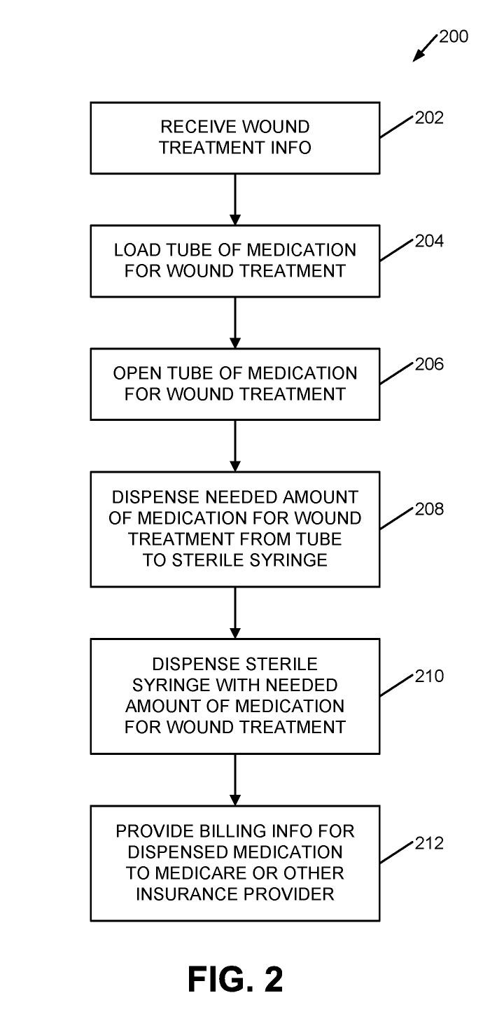

FIG. 2 illustrates an implementation of an example method 200 for dispensing a tube of medication from a medication dispensing device (“dispenser”) according to the present disclosure.

In some implementations, the method 200 comprises receiving 202 medication treatment information, such as a dosage amount, treatment area (e.g., wound area) information, and/or any other suitable information with respect to the tube of medication and/or the medication treatment.

In some implementations, receiving 202 medication treatment information includes receiving any of the details described above with respect to the dispenser 102 receiving medication treatment information 104.

In some implementations, receiving 202 medication treatment information may include receiving information from a Pyxis™ or other medication coordination device 112. In some implementations, receiving 202 medication treatment information may include receiving medication treatment information from any other suitable device.

In some implementations, receiving 202 medication treatment information may include receiving information in any other suitable manner.

In some implementations, the method 200 comprises loading 204 a tube of medication for treatment, such as treatment of a wound, other injury, or any other medicinal treatment.

In some implementations, loading 204 a tube of medication for treatment includes any of the details described above with respect to the dispenser 102 loading or being loaded with a tube of medication 106.

In some implementations, the method 200 comprises opening 206 the tube of medication for treatment.

In some implementations, opening 206 the tube of medication for treatment includes any of the details described above with respect to the dispenser 102 opening a tube of medication 106.

In some implementations, the method 200 comprises dispensing 208 a determined amount of the medication for treatment from the tube into a sterile syringe.

In some implementations, dispensing 208 a desired amount of the medication for treatment from the tube to a sterile syringe includes any of the details described above with respect to the dispenser 102 dispensing a desired amount of medication from a loaded tube of medication 106 to a sterile syringe 108.

In some implementations, the method 200 comprises dispensing 210 the sterile syringe for treatment with the medication.

In some implementations, dispensing 210 the sterile syringe for treatment with the medication includes any of the details described above with respect to the dispenser 102 dispensing a sterile syringe of dispensed medication 108.

In some implementations, the method 200 comprises providing 212 billing information for the dispensed medication, for example to Medicare or other insurance (e.g., medical insurance) provider or to any other suitable payer or recipient.

In some implementations, providing 212 billing information for the dispensed medication includes any of the details described above with respect to the dispenser 102 providing billing information for the dispensed medication 110.

In some implementations, the order of the above-described steps 202-212 may be changed or different according to the present disclosure. For example, in some implementations, step 204 or step 206 may be performed before step 202 according to the present disclosure. In some implementations, there may be any other suitable variation of the order of the above-described steps 202-212 according to the present disclosure.

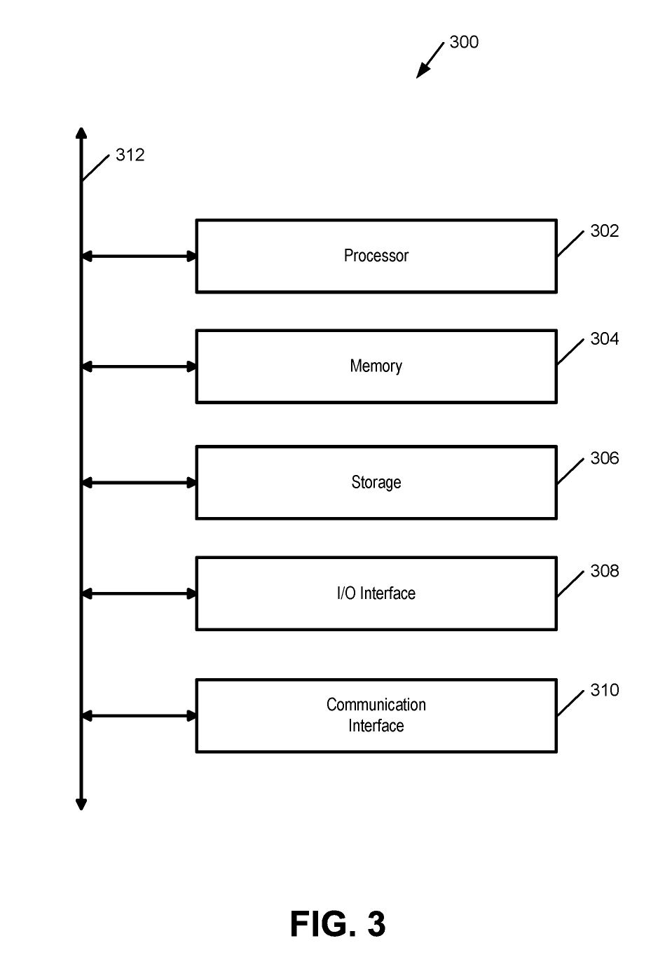

FIG. 3 illustrates an example computer system 300, which may be used with some implementations of the present disclosure. This disclosure contemplates any suitable number of computer systems 300.

This disclosure contemplates computer system 300 taking any suitable physical form. In some implementations, as an example and not by way of limitation, computer system 300 may be an embedded computer system, a system-on-chip (SOC), a single-board computer system (SBC) (such as, for example, a computer-on-module (COM) or system-on-module (SOM)), a desktop computer system, a laptop, an interactive kiosk, a mainframe, a mesh of computer systems, a mobile telephone, a personal digital assistant (PDA), a server, or a combination of two or more of these.

In some implementations, where appropriate, computer system 300 may include one or more computer systems 300; be unitary or distributed; span multiple locations; span multiple machines; or reside in a cloud, which may include one or more cloud components in one or more networks.

In some implementations, where appropriate, one or more computer systems 300 may perform without substantial spatial or temporal limitation one or more steps of one or more methods described or illustrated herein. In some implementations, as an example and not by way of limitation, one or more computer systems 300 may perform in real time or in batch mode one or more steps of one or more methods described or illustrated herein. In some implementations, one or more computer systems 300 may perform at different times or at different locations one or more steps of one or more methods described or illustrated herein, where appropriate.

In some implementations, computer system 300 includes a processor 302, memory 304, storage 306, an input/output (I/O) interface 308, a communication interface 310, and a bus 312. Although this disclosure describes and illustrates a particular computer system having a particular number of particular components in a particular arrangement, this disclosure contemplates any suitable computer system having any suitable number of any suitable components in any suitable arrangement.

In some implementations, processor 302 includes hardware for executing instructions, such as those making up a computer program. In some implementations, as an example and not by way of limitation, to execute instructions, processor 302 may retrieve (or fetch) the instructions from an internal register, an internal cache, memory 304, or storage 306; decode and execute them; and then write one or more results to an internal register, an internal cache, memory 304, or storage 306.

In some implementations, processor 302 may include one or more internal caches for data, instructions, or addresses. The present disclosure contemplates processor 302 including any suitable number of any suitable internal caches, where appropriate. In some implementations, as an example and not by way of limitation, processor 302 may include one or more instruction caches, one or more data caches, and one or more translation look-aside buffers (TLBs).

In some implementations, instructions in the instruction caches may be copies of instructions in memory 304 or storage 306, and the instruction caches may speed up retrieval of those instructions by processor 302.

In some implementations, data in the data caches may be copies of data in memory 304 or storage 306 for instructions executing at processor 302 to operate on; the results of previous instructions executed at processor 302 for access by subsequent instructions executing at processor 302 or for writing to memory 304 or storage 306; or other suitable data.

In some implementations, the data caches may speed up read or write operations by processor 302. In some implementations, the TLBs may speed up virtual-address translation for processor 302.

In some implementations, processor 302 may include one or more internal registers for data, instructions, or addresses. The present disclosure contemplates processor 302 including any suitable number of any suitable internal registers, where appropriate. Where appropriate, processor 302 may include one or more arithmetic logic units (ALUs); be a multi-core processor; or include one or more processors 302. Although this disclosure describes and illustrates a particular processor, this disclosure contemplates any suitable processor.

In some implementations, memory 304 includes main memory for storing instructions for processor 302 to execute or data for processor 302 to operate on. In some implementations, as an example and not by way of limitation, computer system 300 may load instructions from storage 306 or another source (such as, for example, another computer system 300) to memory 304.

In some implementations, processor 302 may then load the instructions from memory 304 to an internal register or internal cache. In some implementations, to execute the instructions, processor 302 may retrieve the instructions from the internal register or internal cache and decode them.

In some implementations, during or after execution of the instructions, processor 302 may write one or more results (which may be intermediate or final results) to the internal register or internal cache. In some implementations, processor 302 may then write one or more of those results to memory 304.

In some implementations, processor 302 executes only instructions in one or more internal registers or internal caches or in memory 304 (as opposed to storage 306 or elsewhere) and operates only on data in one or more internal registers or internal caches or in memory 304 (as opposed to storage 306 or elsewhere).

In some implementations, one or more memory buses (which may each include an address bus and a data bus) may couple processor 302 to memory 304. In some implementations, bus 312 may include one or more memory buses, as described below.

In some implementations, one or more memory management units (MMUs) reside between processor 302 and memory 304 and facilitate accesses to memory 304 requested by processor 302.

In some implementations, memory 304 includes random access memory (RAM). In some implementations, this RAM may be volatile memory, where appropriate.

In some implementations, where appropriate, this RAM may be dynamic RAM (DRAM) or static RAM (SRAM). Moreover, in some implementations, where appropriate, this RAM may be single-ported or multi-ported RAM. The present disclosure contemplates any suitable RAM.

In some implementations, memory 304 may include one or more memories 304, where appropriate. Although this disclosure describes and illustrates particular memory, this disclosure contemplates any suitable memory.

In some implementations, storage 306 includes mass storage for data or instructions. In some implementations, as an example and not by way of limitation, storage 306 may include an HDD, a floppy disk drive, flash memory, an optical disc, a magneto-optical disc, magnetic tape, or a Universal Serial Bus (USB) drive or a combination of two or more of these.

In some implementations, storage 306 may include removable or non-removable (or fixed) media, where appropriate. In some implementations, storage 306 may be internal or external to computer system 300, where appropriate. In some implementations, storage 306 is non-volatile, solid-state memory.

In some implementations, storage 306 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 306 taking any suitable physical form.

In some implementations, storage 306 may include one or more storage control units facilitating communication between processor 302 and storage 306, where appropriate. In some implementations, where appropriate, storage 306 may include one or more storages 306. Although this disclosure describes and illustrates particular storage, this disclosure contemplates any suitable storage.

In some implementations, I/O interface 308 includes hardware, software, or both providing one or more interfaces for communication between computer system 300 and one or more I/O devices. In some implementations, computer system 300 may include one or more of these I/O devices, where appropriate.

In some implementations, one or more of these I/O devices may enable communication between a person and computer system 300. In some implementations, as an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these.

In some implementations, an I/O device may include one or more sensors. This disclosure contemplates any suitable I/O devices and any suitable I/O interfaces 308 for them.

In some implementations, where appropriate, I/O interface 308 may include one or more device or software drivers enabling processor 302 to drive one or more of these I/O devices. I/O interface 308 may include one or more I/O interfaces 308, where appropriate. Although this disclosure describes and illustrates a particular I/O interface, this disclosure contemplates any suitable I/O interface.

In some implementations, communication interface 310 includes hardware, software, or both providing one or more interfaces for communication (such as, for example, packet-based communication) between computer system 300 and one or more other computer systems 300 or one or more networks.

In some implementations, as an example and not by way of limitation, communication interface 310 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 310 for it.

In some implementations, as an example and not by way of limitation, computer system 300 may communicate with an ad hoc network, a personal area network (PAN), a local area network (LAN), a wide area network (WAN), a metropolitan area network (MAN), or one or more portions of the Internet or a combination of two or more of these.

In some implementations, one or more portions of one or more of these networks may be wired or wireless. In some implementations, as an example, computer system 300 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these.

In some implementations, computer system 300 may include any suitable communication interface 310 for any of these networks, where appropriate. In some implementations, communication interface 310 may include one or more communication interfaces 310, where appropriate. Although this disclosure describes and illustrates a particular communication interface, this disclosure contemplates any suitable communication interface.

In some implementations, bus 312 includes hardware, software, or both coupling components of computer system 300 to each other. In some implementations, as an example and not by way of limitation, bus 312 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCI-X) bus, a serial advanced technology attachment (SATA) bus, a Video Electronics Standards Association local (VLB) bus, or another suitable bus or a combination of two or more of these.

In some implementations, bus 312 may include one or more buses 312, where appropriate. Although this disclosure describes and illustrates a particular bus, this disclosure contemplates any suitable bus or interconnect.

Herein, reference to a computer-readable storage medium encompasses one or more non-transitory, tangible computer-readable storage media possessing structure. In some implementations, as an example and not by way of limitation, a computer-readable storage medium may include a semiconductor-based or other integrated circuit (IC) (such, as for example, a field-programmable gate array (FPGA) or an application-specific IC (ASIC)), a hard disk, an HDD, a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a SECURE DIGITAL card, a SECURE DIGITAL drive, or another suitable computer-readable storage medium or a combination of two or more of these, where appropriate.

Herein, reference to a computer-readable storage medium excludes any medium that is not eligible for patent protection under 35 U.S.C. § 101. Herein, reference to a computer-readable storage medium excludes transitory forms of signal transmission (such as a propagating electrical or electromagnetic signal per se) to the extent that they are not eligible for patent protection under 35 U.S.C. § 101.

This disclosure contemplates one or more computer-readable storage media implementing any suitable storage. In some implementations, a computer-readable storage medium implements one or more portions of processor 302 (such as, for example, one or more internal registers or caches), one or more portions of memory 304, one or more portions of storage 306, or a combination of these, where appropriate.

In some implementations, a computer-readable storage medium implements RAM or ROM. In some implementations, a computer-readable storage medium implements volatile or persistent memory.

In some implementations, one or more computer-readable storage media embody software. Herein, reference to software may encompass one or more applications, bytecode, one or more computer programs, one or more executables, one or more instructions, logic, machine code, one or more scripts, or source code, and vice versa, where appropriate.

In some implementations, software includes one or more application programming interfaces (APIs). This disclosure contemplates any suitable software written or otherwise expressed in any suitable programming language or combination of programming languages.

In some implementations, software is expressed as source code or object code. In some implementations, software is expressed in a higher-level programming language, such as, for example, C, Perl, or a suitable extension thereof. In some implementations, software is expressed in a lower-level programming language, such as assembly language (or machine code).

In some implementations, software is expressed in JAVA. In some implementations, software is expressed in Hyper Text Markup Language (HTML), Extensible Markup Language (XML), or other suitable markup language.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure. For example. it will apparent to one of ordinary skill in the art that the invention may be used with any electronic network service, even if it is not provided through a website.

Any computer-based system that provides networking functionality can be used in accordance with the present invention even if it relies, for example, on e-mail, instant messaging or other forms of peer-to-peer communications, and any other technique for communicating between users. The invention is thus not limited to any particular type of communication system, network, protocol, format or application.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a tangible computer readable storage medium or any type of media suitable for storing electronic instructions, and coupled to a computer system bus. Furthermore, any computing systems referred to in the specification may include a single processor or may be architectures employing multiple processor designs for increased computing capability.

While the foregoing processes and mechanisms can be implemented by a wide variety of physical systems and in a wide variety of network and computing environments, the server or computing systems described below provide example computing system architectures for didactic, rather than limiting, purposes.

The present invention has been explained with reference to specific embodiments. For example, while embodiments of the present invention have been described as operating in connection with a network system, the present invention can be used in connection with any communications facility that allows for communication of messages between users, such as an email hosting site. Other embodiments will be evident to those of ordinary skill in the art. It is therefore not intended that the present invention be limited, except as indicated by the appended claims.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

The present disclosure encompasses all changes, substitutions, variations, alterations, and modifications to the example embodiments herein that a person having ordinary skill in the art would comprehend.

The figures, including any drawings and photographs, included herewith may represent one or more implementations of the method for dispensing medication.

Details shown in the figures, such as dimensions, descriptions, etc., are exemplary, and there may be implementations of other suitable details according to the present disclosure.

Reference throughout this specification to “an embodiment” or “implementation” or words of similar import means that a particular described feature, structure, or characteristic is comprised in at least one embodiment of the present invention. Thus, the phrase “in some implementations” or a phrase of similar import in various places throughout this specification does not necessarily refer to the same embodiment.

Many modifications and other embodiments of the inventions set forth herein will come to mind to one skilled in the art to which these inventions pertain having the benefit of the teachings presented in the foregoing descriptions and the associated drawings.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

While operations may be depicted in the drawings in a particular order, this should not be understood as requiring that such operations be performed in the particular order shown or in sequential order, or that all illustrated operations be performed, to achieve desirable results.

1. A method for dispensing medication from a dispenser, wherein the dispenser is configured to receive medication treatment information, load a tube of a medication, open the tube, dispense the medication from the tube into a sterile syringe, dispense the sterile syringe with the medication, and provide billing information for the medication, the method comprising:
receiving medication treatment information;
loading a tube of medication in the dispenser;
opening the tube of the medication in the dispenser and keeping the medication uncompromised;
dispensing a determined amount of the medication from the tube into a sterile syringe in the dispenser and keeping the medication uncompromised;
dispensing the sterile syringe containing the determined amount of the medication and keeping the medication uncompromised; and
providing billing information for the determined amount of the medication dispensed in the sterile syringe.

2. The method of claim 1 wherein the medication treatment information comprises a dosage amount of the medication

3. The method of claim 1 wherein the medication treatment information comprises treatment area information to determine the dosage amount of the medication.

4. The method of claim 3 where the treatment area information comprises information regarding the size of a wound of a patient for wound treatment.

5. The method of claim 1, wherein the determined amount of the medication dispensed into the sterile syringe is based on the medication treatment information.

6. The method of claim 1, wherein the medication treatment information is received from a Pyxis™ device.

7. The method of claim 1, wherein the billing information is provided to payer over a network.
